(19) United States
(12) Patent Application Publication
(10) Pub. No.: US 2024/0133874 A1
(43) Pub. Date: Apr. 25, 2024

(54) IMPROVEMENTS IN OR RELATING TO IMMUNITY PROFILING

(71) Applicant: Fluidic Analytics Limited, Cambridge, Cambridgeshire (GB)

(72) Inventors: Alekszej Morgunov, Tallinn (EE); Simon Morling, Cambridge (GB); Tuomas Knowles, Cambridge (GB)

(73) Assignee: Fluidic Analytics Limited, Cambridge, Cambridgeshire (GB)

(21) Appl. No.: 18/276,998

(22) PCT Filed: Feb. 21, 2022
(86) PCT No.: PCT/GB2022/050461

§ 371 Date: Aug. 11, 2023

(30) Foreign Application Priority Data
Feb. 22, 2021 (GB) 2102491.4

(51) Int. Cl. G01N 33/536; G01N 33/53; G16H 10/40; G16H 50/30

(52) U.S. Cl. CPC G01N 33/536; G01N 33/5302; G16H 10/40; G16H 50/30

(57) ABSTRACT

A system is provided for developing a predictive immunity profile on the basis of the quantitative analysis of one or more samples from an individual. The system comprises: a device configured to perform quantitative analysis of the interaction between one or more target species and one or more probes in solution on a fluid sample to provided quantitative analysis data; a data store storing: personal data relating to at least one individual; at least one model of target/probe interaction; processing circuitry configured to access the data store and identify and retrieve data relevant to the sample; receive quantitative analysis data of the sample from the device; perform analysis to fit the model to the received quantitative analysis data; extrapolate, through the model, to create a predictive immunity profile for the individual, and update the data store with the quantitative analysis data.

The present invention relates to immunity profiling and, in particular, to improvements in the intelligence gathering and knowledge processing leading to improved predictive profiling.

Immunisation programmes have, historically, been rolled out on the basis of population wide modelling and prediction of need on the basis of vulnerability. However, individual immune response characteristics are multidimensional and complex and a blanket immunisation strategy can be neither efficient nor cost effective.

In recent times, many have studied the interactions between target species or biomolecules such as proteins to help develop more sophisticated diagnostics/medical tools for various diseases. Protein-protein interactions (PPIs) form the basis of many biologically and physiologically relevant processes including: protein self-assembly; protein-aggregation; antibody-antigen recognition; muscle contraction and cellular communication. Nevertheless, studying protein-protein interactions, especially under physiological conditions in complex media, remains challenging. Current techniques, such as an enzyme-linked immunosorbent assay (ELISA) bead-based multiplex assay and surface plasmon resonance (SPR) spectroscopy, rely on immobilisation of one binding partner. These techniques include potential non-specific interactions with the surface, which can cause false positive results and the Hook/Prozone effect, which causes false-negative results, thereby allowing semi-quantitative analysis only.

Currently all known approaches for clinical protein detection and quantification involve assays relying on surface immobilisation or bead immobilisation, as for example ELISA assays. While ELISA assays can achieve a relatively high sensitivity, they are not capable of determining biophysical parameters describing the binding interaction in solution, and therefore they do not offer a realistic model of the physiologically relevant processes being studied as these all take place in solution.

Alternative strategies have emerged for achieving enhanced sensitivity including the use of an antibody pair for detecting protein molecules and the use of apparatus developed for flow cytometry for reading out relative intensities. However, these assays are still performed on the surface of a bead and therefore encounter the usual disadvantages of surface-based techniques described above. In addition, these techniques are often time consuming.

Machine learning algorithms have recently been used in protein-protein interactions studies and in particular for the study of protein functions and pathways involved in different biological processes, as well as for understanding the cause and progression of diseases. Some experimental techniques have been employed for the identification of PPIs but these are limited to a binary output and there is still a gap in identifying, analysing and predicting the biophysical properties in PPIs to provide meaningful outcomes for a patient.

Thus, there is a requirement to provide an in-solution platform that can be used to collate and analyse quantitative data relating to target/probe interactions and to recommend outcomes to an individual based on models of target/probe interactions combined with these quantitative analyses of measured biophysical properties between biomolecule interactions, such as biomarkers and their specific targets in body fluids in a fully quantitative manner.

Furthermore, understanding the nuances of the increased breadth of biomedical data available is enabling a more personalised approach to immunity profiling so that immunisation programmes can be tailored to take into account the patient's individual biomedical data.

According to the present invention there is provided a system for developing a predictive immunity profile on the basis of the quantitative analysis of one or more samples from an individual, the system comprising: a device configured to perform quantitative analysis of the interaction between one or more target species and one or more probes in solution on a fluid sample to provided quantitative analysis data; a data store storing: personal data relating to at least one individual; at least one model of target/probe interaction; processing circuitry configured to access the data store and identify and retrieve data relevant to the sample; receive quantitative analysis data of the sample from the device; perform analysis to fit the model to the received quantitative analysis data; extrapolate, through the model, to create a predictive immunity profile for the individual, and update the data store with the quantitative analysis data.

The system obtains quantitative measurement of characteristics of interactions between one or more probes and one or more target species which provide a characterisation of the immune response of an individual. This characterisation of the immune response under a first scenario is then used to infer the properties of the immune response under a second scenario without direct measurement of the second scenario. The second scenario may be measurable, but there is a desire to infer the immune response without direct measurement. For example, the second scenario may be at a later time and the inference of the immune response may be desired ahead of time.

The probe may be a single probe, but it is often a combination of multiple probes and the data store therefore includes models relating to the interaction of each probe with the target species. The target species is often an antibody, but it could be an antigen, receptor or any other biomolecular component of interaction with a probe.

In some embodiments, there may be one or more target species interacting with one or more probes.

In some embodiments, the system may obtain quantitative measurement of characteristics of interactions between one or more probes and one or more target species simultaneously, in order to provide a characterisation of the immune response of an individual.

In some embodiments, the system may obtain quantitative measurement of characteristics of interactions between one or more probes and one or more target species in a sequential manner, in order to provide a characterisation of the immune response of an individual.

Model based extrapolation from the measured data points is used to create a predictive immunity profile that is specific to an individual. In the context of models that describe the decay of immune response to a specific antigen, set of antigens or disease, it is acknowledged that decay of immunity is not uniform across a species and different individuals' level of immunity may decay at a different rate for a number of different reasons. Therefore it is no longer sufficient to apply a blanket immunity profile to an immunisation across an entire population.

By storing personal data from an individual and by updating that personal data on the basis of each measurement taken, an increasingly personalised predictive immunity profile is created for the individual.

A data store is an ever-developing repository of information drawn from various sources and involved in all system activities. Even if it commences with only low confidence experimental data, this is sufficient to add some value and, as the device undertakes quantitative analysis of samples, the additional data gleaned from this analysis is used to augment the data store.

The predictive immunity profile may include a date on which the patient's immunity is predicted to fall below a predetermined threshold. It is clear that immunity does not decay equally for all vaccinated individuals. As a result, the date on which a booster vaccination is required will vary from individual to individual. Therefore, some individuals may require a booster vaccination earlier than recommended date based on the general population. This is a temporal extrapolation. The behaviour of the individual's immune system is recorded through one or more quantitative analyses from the device and this is combined with personal data about the individual, including the date of the vaccination or disease recovery. This is used to extrapolate in time to predict the behaviour of the individual's immune system at a future point in time.

The predictive immunity profile may include a confidence level that the individual will exhibit immunity to a different disease strain from the strain on which the vaccine was based.

The predictive immunity profile may include a confidence level that the individual will exhibit immunity to a different disease strain from the strain that the individual contracted.

Viruses change over time through the process of antigenic drift to create mutated viral forms, or different variants of a virus. A vaccine is developed on the basis of the most prevalent strain or strains of the virus at the time of development. However, over time, different mutations may take on a wider prevalence and immunity to one strain following vaccination against another strain becomes relevant. In this scenario, the device measures the individual's immunity profile in relation to a reference strain which may be the strain that the vaccine was developed against. The model is then used to predict the confidence level, or percentage likelihood, that the individual will also exhibit immunity against a different variant. This can be termed a longitudinal extrapolation in that it extrapolates from a strain against which a vaccine has been applied and considers the confidence level that immunity also exists against a new strain.

The data store may hold data on multiple individuals. The more data in terms of both the number of data points relating to an individual and also the number of individuals' data being included in the data store, the more nuanced and accurate the modelling can be and therefore the higher the confidence in the predicted immunity profile for the individual.

The data held in the data store on each individual may include one or more of the following: medical records, age, gender, weight, ethnicity, genetic information, vaccination history, disease state, disease severity, identity of medication prescribed and corresponding dosage regimen.

The more information that is available about each individual, the more informed the model can be and therefore the greater the certainty regarding the accuracy of the predicted immunity profile that is output by the system.

The sample may be a bodily fluid. In particular, the sample may be, or may be derived from blood including serum and plasma or it may be CSF, saliva, sweat, faeces or urine. In some embodiments, the bodily fluid may be mucus, synovial fluid, amniotic fluid or milk.

The data store may further comprise stratification labels and wherein the processing circuitry is further configured to use the stratification labels when performing analysis to fit the received quantitative analysis data to the model.

Stratification labels enable individuals to be classified into cohorts on the basis of one or more characteristic within their personal data. These cohorts provide an additional nuance to the model which can be used to bolster or stand in place of a priori knowledge about an individual.

For example, the age of the individual can impact the immunity profile of an individual. Typical data for the individual would include the date of vaccination and therefore the time between vaccination and sample provision. This allows the immunity profile to be extrapolated forward in time to predict when immunity will fall below a predetermined acceptable threshold when the individual should be re-immunised. The stratification label pertaining to the individual's age can also inform the model on the basis of the observed profile to other individuals of a similar age.

The individual's age is a very simple example of the stratification labels. It will be appreciated that various factors may interact in complex relationships and these can be captured in stratification labels.

The data store may further comprise a general model and wherein the processing circuitry is further configured to use the general model when performing analysis to fit the received quantitative analysis to the model.

A general model relates to, for example, protein-protein interactions themselves. The source of the model may include any experimental or patient data either from the system itself, as proprietary data sets or from third party library data sets. The creation of a model general to the protein-protein interactions themselves also enables missing data to be predicted by interpolation of existing data. An accuracy score of interpolated or predicated data points will reflect the nature of these data points.

The circuitry configured to perform said analysis may comprise a machine learning algorithm.

The processing circuitry may be further configured to update the personal data relating to the individual's sample analysed. This provides the closure of the feedback loop at the individual level. The individual's personal data, held within the data store, is augmented with the new quantitative analysis of the sample. This data is stored with the individual's record, along with processed outcomes and other meta-data derived from the quantitative analysis of the sample.

In relation to the data sources deployed with the system, the data relating to antibody/probe interactions may include anonymised data from individuals and experimental data.

The quantitative analysis of the sample may include a measurement of affinity of a target/probe interaction.

The quantitative analysis of the sample may include a measurement of the concentration of a target species within the sample.

The quantitative analysis of the sample may include analysis of the heterogeneity of the sample.

The heterogeneity of the sample may include, but is not limited to, the presence of, and/or extent of isoforms, post translational modifications, different stoichiometry, extra binding partners, splice isoforms. The quantitative analysis of the sample may further include analysis of the charge, mobility, hydrodynamic radius, amino acid content within a protein, fluorescence of a protein.

The device may comprise a microfluidic network configured to enable combination and distribution of a sample fluid and an auxiliary fluid to create a distributed sample and subsequent division of the distributed sample into two or more parts and measurement of at least one of the parts.

The distribution may be created by one or more of diffusion, electrophoresis or magnetophoresis, thermophoresis, chromatography and isoelectric focusing.

The device may be configured to divide the distributed sample into more than two parts and measurement is carried out on each divided part. Various different chromatographic techniques may also be applicable including, but not limited to size exclusion chromatography and reverse phase chromatography.

Each predicted result generated by the system may have an associated accuracy score. Moreover, the step of updating the data store may include update the accuracy score.

The accuracy score will inform the algorithm as to the source of the data, for example, whether the data was obtained using an analogous device to that included in the system or a different device.

The predicted immunity profile generated by the system may have an associated accuracy score. The accuracy score in the predicted immunity profile takes into account both the fundamental or aleatoric error relating to the accuracy of the data and the epistemic error relating to how close the new data point is to the existing model as developed from the training data set.

The data relating to target/probe interactions may include predicted data based on adjacent data. Where the accuracy score of a subset of the data is sufficiently high, and therefore confidence in the accuracy of that data is sufficiently high, the machine learning algorithm is configured to provide predictions of expected results that lie adjacent in the data space to pre-existing data points with high confidence.

The quantitative analysis of the device may include a fluorescence measurement. The fluorescence may be intrinsic to one or more of the species or it may arise from a fluorescent label applied to a target species or probe. For example, an antibody, antigen or receptor may be labelled with a fluorescent label which may emit when specific binding has taken place. The binding that causes the fluorescence may be simply between the labelled component and a second component. Alternatively, the interaction may be more complex, including three or more components, at least one of which may be labelled.

The invention will now be described, by way of example only, with reference to the accompanying drawings in which:

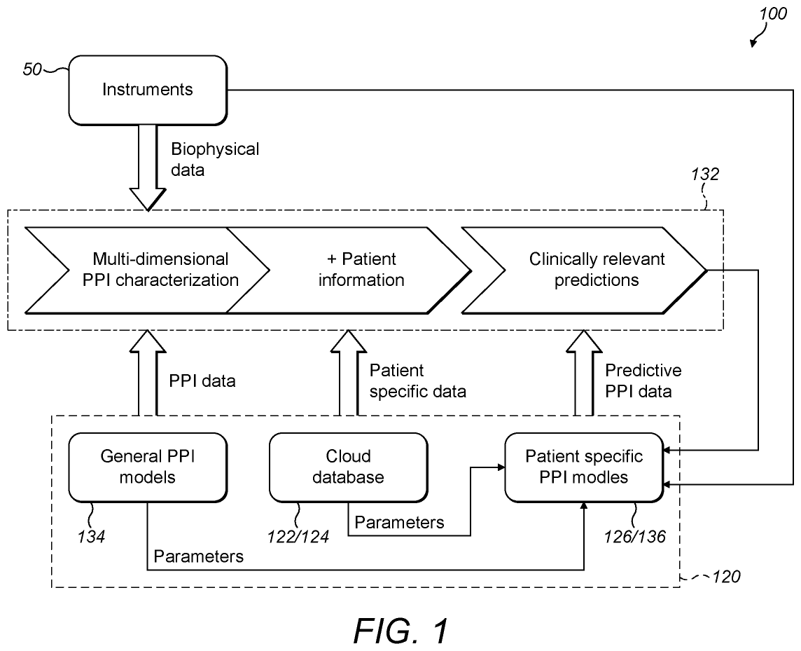

FIG. 1 shows a flow diagram showing the workflows of the system of the present invention;

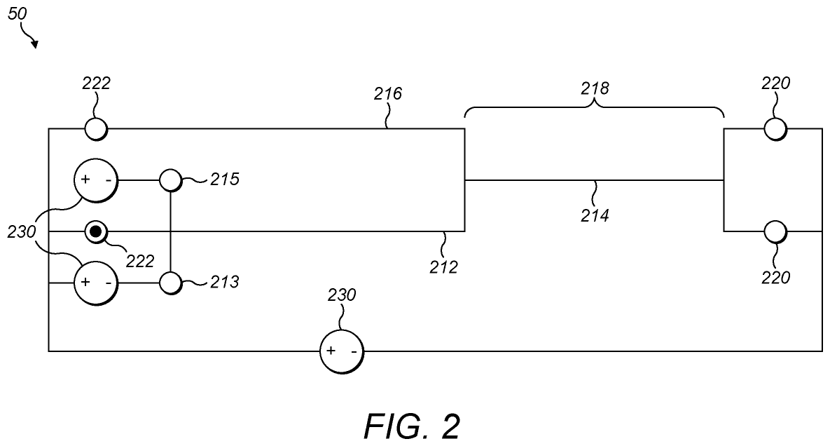

FIG. 2 is a schematic of a microfluidic device provided within the system of the present invention;

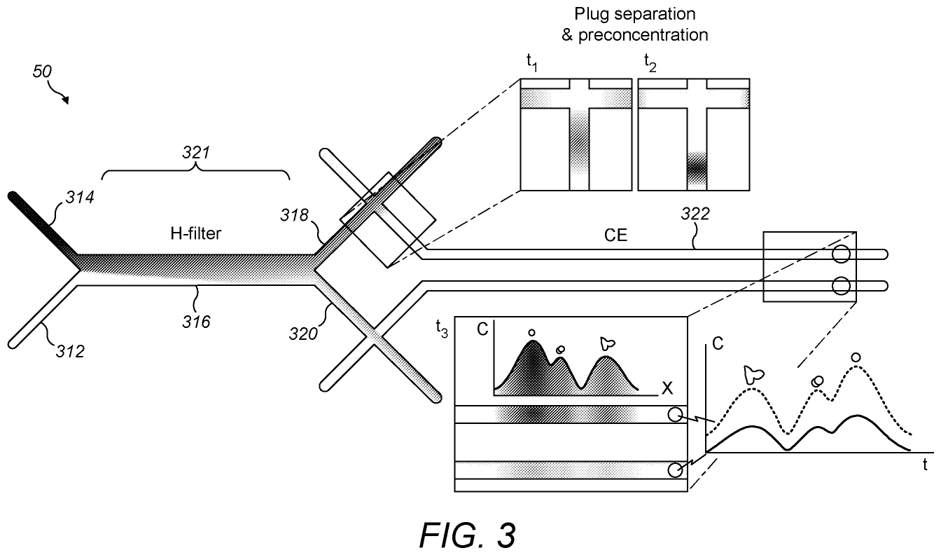

FIG. 3 is a schematic of an alternative microfluidic device provided within the system of the present invention;

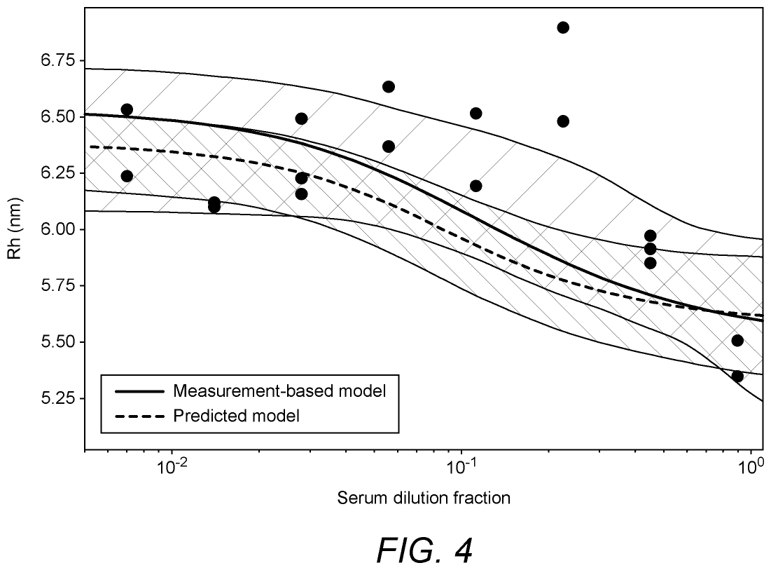

FIG. 4 shows experimental data from an example using neutralisation data for immunity profiling;

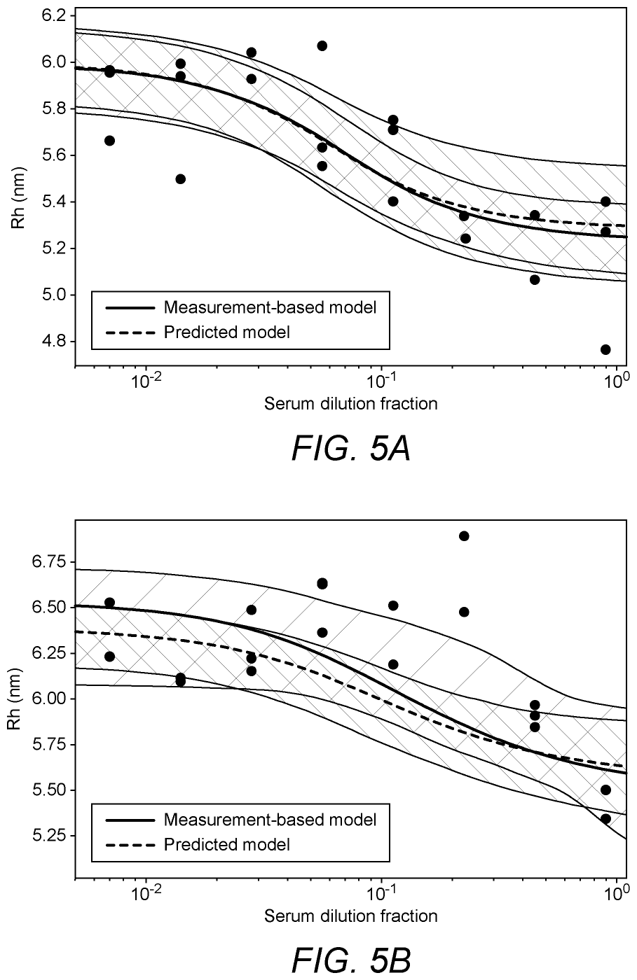

FIGS. 5A and 5B show control data associated with the experimental data of FIG. 4;

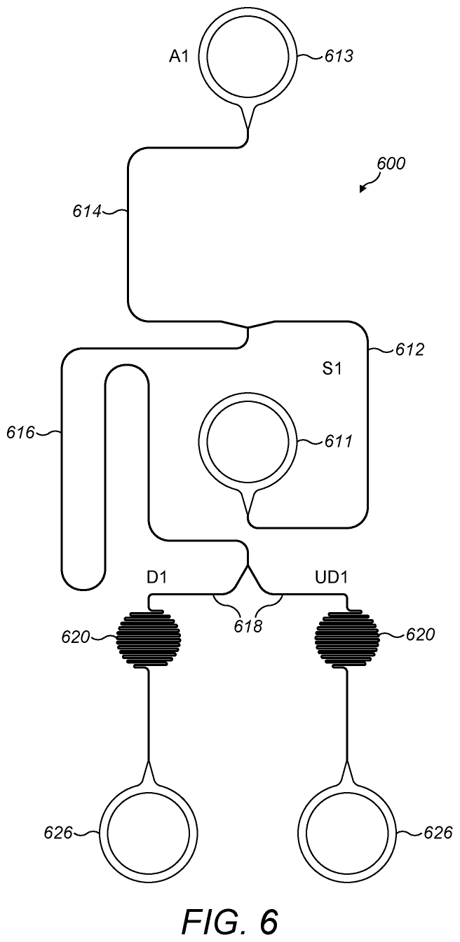

FIG. 6 shows a flow device for measuring at least one biophysical property of one or more bimolecular components in a heterogeneous sample; and

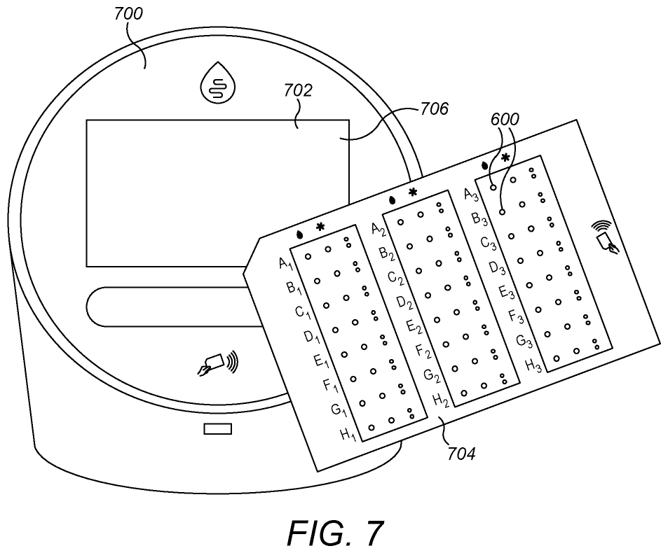

FIG. 7 shows an instrument that forms part of the system of the present invention.

FIG. 1 shows the workflow achieved on the system 100 of the present invention. The system 100 includes a data store 120 which can include proprietary knowledge database 122, third party databases 124 including open source general data pertaining to protein-protein interactions or other relevant bio-macromolecular interactions, sometimes referred to as General Models. In addition, the data store 120 includes individual personal data 126 some of which may be generated on the device 50 (shown and described in more detail below with reference to FIGS. 2 and 3). Other personal data includes at least the identity of the individual and one or more of the following related data: medical records, age, gender, ethnicity, vaccination history, disease state, medication taken and respective dosage regimens observed. The data store 120 may be a single data store or it may include a plurality of sub stores each storing data from a specific source. These sub stores may be physically co-located with the device 50 or they may be distributed, in particular, cloud based. Regardless of their physical location, they are functionally linked and are therefore referred to generally as the data store 120.

The system 100 also includes a machine learning algorithm 132 which accesses at least one general model 134 pertaining to bio-macromolecular interactions. The machine learning algorithm 132 deploys a plurality of different algorithms each selected for use with a model. There is at least one general model 134 and one specific model 136 of an immune profile.

When a sample is obtained from an individual, in addition accessing the general model 134 pertaining to the relevant target/probe interactions, the machine learning algorithm 132 also accesses the data store 120 to obtain personal data 126 pertaining to the individual. This data may include previous quantitative data from the device 50 from previous quantitative analyses. In addition, this may include personal data such as the individual's age, weight, gender, medication regimen and other relevant risk factors. Furthermore, the machine learning algorithm 132 will access a specific model 136 pertaining to a characterisation of immune response. The device 50 undertakes the sample preparation and flows the sample through the device 50, producing the lateral distribution and taking quantitative readings leading to a measurement of, for example, affinity, concentration or heterogeneity of the sample.

The quantitative data obtained from the device 50 is then combined with the general model of the probe/target interaction data, the personal data of the individual and the specific model relating to the immune response of interest. This combination of multi-dimensional protein-protein interaction characterisation with personal data for the individual and specific model of the immune response of interest, enables an inference to be made about a different scenario from the measured scenario. This inference or extrapolation from the measured data can be provided as a clinically relevant output to be given to the individual.

For example, where the immune response of an individual has been characterised, through measurement of affinity, to indicate the extent of the decay of neutralisation capacity, this gives a snap shot in time of the individual's level of immunity to a specific virus or other pathogen. The system is configured, through the combination of this measured data with the general model, specific model of decay of immunity against that virus/pathogen over time and personal data to give a temporal extrapolation of the individual's immunity. Additionally or alternatively, the system can be configured to use general modelling of different variants of a virus or pathogen, to predict an individual's level of immunity against a new variant of the virus or pathogen which is either different from the variant against which a vaccine was initially developed and/or different from the variant with which the individual has previously been infected.

This characterisation of the individual's immune function can be provided, as a clinically relevant output to the individual or their clinician in terms of a date at which the individual's immunity is predicted to fall below a predetermined threshold, making repeat immunisation advisable. Therefore, the system as disclosed herein may be particularly useful for clinicians to recommend an accurate date and/or time for a booster vaccination of an individual. In particular, the clinicians may be able to determine when an individual requires a booster vaccination at an earlier date than the recommended date for the general population. Additionally, if there are multiple versions of the vaccine developed against different variants, the individual or the clinicians may be able to determine the most appropriate choice of the vaccine.

In a further example, the immune response of an individual can be characterised, through measurement of affinity, to indicate the extent of immunity against a specific variant of a virus. The system is configured, through the combination of this measured data with the general model, specific models of various viral strains or variant and the individual's personal data to give a confidence level of the individual's immunity against a different strain from that against which the individual has been immunised or has recovered from contracting. In this example, the output for the individual will be a percentage. In this example, the initial data may be neutralisation data based on measurement of the individual's response to the wild type virus. This may be combined with independent data on the mutant assay that is not specific to an individual. These may be combined to extrapolate and create a predicted immunity profile for the different mutant in the individual. This would be relevant, for example, in assessing the likelihood of contracting a new mutated strain of a virus when the individual has previously been infected with and recovered from the wild type or reference strain of the virus.

### Machine Learning Algorithm

The term machine learning algorithm is used to refer generally to a combination of numerous different algorithms each of which is selected for use with the characterisation of an immune response and the prediction or extrapolation of the measured scenario into one or multiple unmeasured scenario. Different algorithms will be appropriate for general models of probe/target interactions, for specific models of characterisation of immune response and for affinity measurement.

For example, for the general model a fully connected deep neural network, recurrent neural network, convolutional neural network or self-attention based architectures, such as transformer based architectures, may be deployed. Representational learning may be used to generate embeddings of sequences and structures of biomacromolecules. These algorithms may be combined with classifier or regressor systems as appropriate. Examples of classifiers that may be appropriate for a general include random forests, gradient boosting machines, Gaussian processes or multilayer perceptrons. A single classifier may be deployed. However, in some embodiments the stacking of classifiers may be achieved. In order to achieve an effective stacking of classifiers, the classifiers are trained to predict the error in the output, rather than the output itself. A combination of Gaussian process and multilayer perceptrons is effective in this context.

The stacking of classifiers is advantageous in this context because the field of probe/target, in particular, protein-protein interactions is complex and the data sets are comparatively small.

In order to introduce the data into the machine learning algorithm it is first necessary to vectorise the bio-macromolecule of interest, for example, a protein, so that the complex structure of the protein can be represented as a numerical vector. This vector can then be ingested by the classifier and thus processed through the machine learning algorithm. This allows the data to be used, initially, to train the machine learning algorithm and, in combination with many other similarly vectorised bio-macromolecular data, to develop a prediction of new quantitative analysis of the interaction of that bio-macromolecule.

The specific models, relating to the modelling of immune response, a tabular data set with associated data transformation and encoding for algorithms can be deployed. Similar classifiers or regressors as described above with reference to the general model may be deployed. In addition, specific models will be informed additionally by information from the general model.

FIG. 2 shows an example of a device 50 that can be incorporated into the system 100. FIG. 2 shows a device 50 configured to provide separation and analysis of a plurality of components in a heterogeneous sample. The device incorporates two sections: a capillary electrophoresis section and an H-filter 218. Although, in the illustrated embodiment, the capillary electrophoresis section precedes the H-filter, it will be appreciated, that the order can be switched so that the H-filter is deployed first. In that configuration, a capillary electrophoresis module can be applied to each of the outputs of the H-filter so that there are as many capillary electrophoresis modules as there are outputs of the H-filter.

The device 50 includes the constituent parts of an H-filter 218 with a sample channel 212 and a buffer channel 216 through which the sample and a buffer or auxiliary fluid can be introduced. The sample channel 212 and the buffer channel 216 terminate at a distribution channel 214 that is elongate is a first direction.

As a result of the elongate configuration of the channels, when a sample flows along the sample channel 212, into and through the distribution channel 214, a distribution of the components in a second direction, substantially perpendicular to the first direction will develop.

The device 50 may include at least one power source 230 configured to provide an electrical field across the distribution channel 214 of the H-filter 218 in order to drive the distribution by electrophoresis.

The H-filter 218 has two outlets 220 and the fluid in the distribution channel 214 is divided between the two outlets. Quantitative analysis of the fluid collected at each of the outlets can be undertaken and data can be compared between the outlets 220. The quantitative analysis will be associated with the regimen under which the lateral distribution was created. Therefore in the device illustrated in FIG. 2, where the power source 230 creates an electrical field across the distribution channel 214 so that the distribution is achieved through electrophoresis, then the quantitative analysis is of the charge on the components within the sample.

Conversely, if the power source 230 in FIG. 2 is not activated, then the distribution in the distribution channel 214 will arise solely via diffusion and the quantitative analysis will be related to the size of the components within the sample.

Alternatively, or additionally, the distribution created in the distribution channel may be achieved by capillary electrophoresis. In addition, the lateral distribution can be created diffusively, electrophoretically, diffusophoretically or thermophoretically.

The device 50 is configured to separate and analyse fluid samples using capillary electrophoresis (CE) separation and diffusive sizing. As shown in FIG. 2, the device 50 comprises an H-Filter 218 with one or more extended inlets 222. Loading of the sample takes place through a sample port 213 into the sample channel 212 and is either achieved via electro-osmotic flow (EOF) or it is pressure-driven. Once the sample has reached the separation channel 214, an electric field is applied across both ends i.e. inlets 222 and outlets 220 of the H-filter 218 to drive the entire distribution channel 214 electro-osmotically. In order to provide control over the sample being supplied to the sample channel 212 there is a sample waste port 215 corresponding to the sample inlet port 213.

As shown in FIG. 2, there is provided at least one power source 230 so that a voltage can be applied to the sample channel 212 and the auxiliary channel 216. FIG. 2 shows exemplary configurations for the voltage supplies 230 and electric connections that can be used to run the device 50 of the present invention. The appropriate selection of the polarity of the power supply 230 will depend on the predicted charge on the components in the sample.

In the embodiment illustrated in FIG. 2, the sample channel 212 and the auxiliary channel 216 are of equal length. The sample channel 212 and the auxiliary channel 216 also have equal cross sectional area. Having the separation channel and the auxiliary channel of equal dimensions simplifies the control of the electro-osmotic flow through the device as the same voltage can be applied across both the sample channel 212 and the auxiliary channel 216, thereby providing substantially equal electro-osmotic flow rates in the sample channel 212 and the auxiliary channel 216.

Moreover, the symmetry between the auxiliary channel 216 and the sample channel 212 ensures equal flow entering the distribution channel 214 and/or throughout the whole H-filter 218. Flow sensors or reference samples (not shown in the accompanied Figures) can be included to determine the bulk flow rate. Reference samples can be introduced into either the separation channel or the auxiliary channel.

Furthermore, the sample can be separated via CE in the separation channel 214 and then can be subjected to diffusive sizing in the H-filter 218. The symmetry the sample channel 212 and the auxiliary channel 216, as well as the constant applied electric field across both channels may provide well-defined flow rates. In some embodiments, the auxiliary capillary may also contain a cross-channel (not shown in the accompanied Figures) for sample loading to enhance symmetry.

The device 50 may also include a sample preparation module (not shown) in which the sample can be prepared ready for introduction into the sample channel 212. The sample preparation module includes a microtitrator to enable the concentration of the sample to be controlled. The sample preparation module also includes temperature and humidity controlled storage conditions so that the sample preparation module can mix and store the sample under conditions and for a time period recommended. The mixture created in the sample preparation module may include ternary or higher order mixtures.

FIG. 3 shows an alternative device 50 for use in the system 100. The version of the device 50 illustrated in FIG. 3 is configured for carrying out a method of determining a biophysical property, for example the diffusion coefficient, of one or more components in a polydisperse sample.

The device 50 comprises an auxiliary channel 312 for an auxiliary fluid, such as buffer, and a sample channel 314 for a fluid comprising the polydisperse sample. The polydisperse sample comprises at least one or more components which may be biomolecules. An auxiliary fluid flow is introduced into a fractionation channel 316. The polydisperse sample comprising one or more components can subsequently be introduced into the fractionation channel 316. The sample and the auxiliary fluids can be combined in the fractionation channel 316 to create a combined flow.

The combined flow is fractionated into two or more fractions 318, 320 by diffusion. Two or more components from each fraction can be separated by creating a distribution of the components within a separation channel 322.

Use of a microfluidic device 50 provides a means to reduce the movement of components to advection (following the flow direction) and diffusion (isotropic). An H-filter 321 is provided as part of microfluidic device 50, as indicated in FIG. 3, to allow two fluid flows of different solutions to join into the fractionation channel 316. The H-filter 321 also has two fractions 318, 320 at one end of the fractionation channel 316 where the combined flows can be split into each fraction 318, 320. The only mixing which occurs between the two solutions in the fractionation channel 316 is due to diffusion perpendicular to the flow direction.

The collection of solution can be done after the fractionation step in two fractions 318, 320. Each fraction contains a proportion of the initial concentration of particles depending on the diffusion coefficient. The diffusion coefficient is related to the size of the particle (hydrodynamic radius).

This information is hidden in a heterogeneous sample, as a direct measurement will give an average of the hydrodynamic radius of the entire sample. Once the different fractions are collected, the components in each fraction can be labelled with an affinity probe and/or an immunoprobe in the solution. This step is preferably not performed prior to the H-filter as the probe will change the size of the particles within the solution.

In a subsequent step, the components in each of the fractions can be separated in solution using capillary electrophoresis in each of the separation channels 322, either with one capillary being used to separate each fraction, or two distinct capillaries being used, one for each fraction. The optical detector records each separated, in solution, component sequentially in each fraction, although two or more capillaries can be observed in parallel where appropriate.

Electrophoresis is a technique for separation of nucleic acids, peptides, and cells. Gel electrophoresis, in which analyte charge-to-size ratio is assessed via retardation in a solid matrix upon the application of an electric field, is the most common technique, though this is not well suited for the study of weak protein association events as the act of matrix sieving itself can disrupt interactions. Capillary Electrophoresis (CE) involves the temporal separation of analytes based on their differential electrophoretic mobility and electroosmotic flow throughout a channel. In Free-Flow Electrophoresis (FFE), the sample moves throughout a planar channel through pressure or displacement-driven flow, and separation upon application of an electric field is perpendicular to the direction of flow. Because FFE is a steady-state technique, injection and separation are performed continuously. Microfluidic Free-Flow Electrophoresis (MFFE), a microfluidic miniaturization of FFE, has the advantage of improving separation resolution by reducing the effect of Joule heating and facile on-line integration with other separation techniques.

The separation of the components using capillary electrophoresis is made using the mobility differences of the two or more components such as different protein complexes with labelled affinity probes and/or immunoprobes. Different peaks should appear in function of the mobility of the different type of biomolecule within the solution.

The separation step may be performed under native conditions to allow an understanding of the component and its environment, including its relationship with other components in a multicomponent mixture. The subsequent analysis may include denaturing and labelling steps to permit accurate identification and characterisation of separated component.

At a point downstream from the separation channels 322, a characteristic of each the two or more components in each fraction can be detected using high resolution or high sensitivity detection techniques such as a fluorescence detection technique by means of a photodiode, photomultiplier or a high resolution camera.

The characteristic of each component in each fraction can be compared in order to determine the biophysical property of each of the two or more components in the polydisperse sample.

Each fraction is collected separately. At this point, the biophysical properties of the components do not need to be extractable. The fractions can then be further processed in a processing step as described below. Some processing can be applied to each fraction, such as labelling, after the fractionation step and prior to the separation step. The labelling of components can be fluorescence labelling such as latent or non-latent labelling or use of affinity probes and/or immunoprobes.

Quantitative labelling procedures, such as the fluorescent labelling procedures described herein, allow the concentration of a component to be directly determined from the recorded analytical signal.

In some cases, non-latent labels may have the same fluorescent intensity if they are attached to the component of interest or not. For example: if approximately 30% of the labels are attached to the component and approximately 70% are unattached to the component, there would be 100% fluorescence. This is the same signal for any attached percentage. Therefore, the signal does not provide any information. With the method of the present invention, the attached and unattached labels would be separated in different separated species. Therefore, the detection of one peak with 30% fluorescence and there shall be a further detection of another peak with 70% fluorescence. Thus, the method of the present invention enables the effective detection of non-latent labels attached to the components and the subsequent analysis of each separated component in each fraction.

Additionally or alternatively, it is also possible to add into each fraction solutes or solvents ions/salts to perform pre-concentration steps i.e. increase the effective concentration of the component in the collected sample.

The added solutes/solvents can affect the molecules or particles or concentrations in each fraction. Therefore, performing this step after fractionation step can help preserve the related biophysical properties of the component, even if the proteins are modified.

Another example would be to use a concentration assay to increase the concentration of the component in each fraction, such as a pull-down assay of immunoprecipitation. The pull-down assay or immunoprecipitation may be performed using magnetic beads for collection. Typically, upon elution from the concentration set up the component would be altered due to denaturation, digestion, or being bound to a capture probe.

Fractions can be separated using a number of high-resolution separation techniques. This is especially important for labelled immunoprobes for example. The separation can be based on, but not limited to, a gradient such as electrophoresis, diffusiophoresis, pH gradient, magnetic field and/or a non-direct separation technique like a labelling affinity technique.

The high-resolution separation of each fraction can be performed in series or in parallel (at the same time). The result of this separation step can be transient, and a measurement is taken at the outlet. Separation fractions can also be collected and then measured later. A high number of very small separation fractions may be collected quickly to keep the separated species separated. The measurement can be done by different technique, Detection of the component in each fraction can be done optically for example using fluorescence (autofluorescence or with help of optional labelling step), absorbance detection techniques, scattering detection techniques, electromagnetically, electrochemically and/or mass measurement.

There may be a detection zone for detecting of the components. The detection can occur downstream from the separation channel. The detection zone may comprise the analytical or detection devices for analysing at least one component in each fraction.

The detection or analytical device is not particularly limited and includes those device that are suitable for use with flow apparatus, and particularly microfluidic devices. A plurality of analytical devices may be provided to determine different physical and chemical characteristics of the component. The analytical devices may be arranged sequentially or in parallel. In some examples, the analytical device can be a fluorimeter or a dry mass measuring device, such as a quartz crystal microbalance.

The separation result from each fraction is compared. This comparison enables the extraction of the biophysical properties, such as the hydrodynamic radius, of each separated component from the fractionation step. In some instances, after appropriate mixing and reaction, the components in each fraction can be compared.

The components can be analysed by calculating the size by cross-correlating diffused and undiffused curves with an optimised scaling factor. The scaling factor can be converted to hydrodynamic radius via finite-element modelling or analytical calculations (depending on the H-filter geometry).

The flow rate of each flow in the sample channel, auxiliary channel, fractionation channel and/or the separation channel can be maintained at a substantially constant level during the fractionation, separation and analysis steps. The fractionation, separation and analysis steps may be undertaken when a stable flow is established in the channels of each section.

The component may be or comprise a polypeptide, a polynucleotide or a polysaccharide. In some embodiments, the component is or comprises a polypeptide. In some embodiments, the component is or comprises a protein. The component may be part of a multicomponent complex. The separation step may therefore be used to at least partially separate the component from other components. For example, the techniques described herein allow for separation based on size or charge-to size ratio, amongst others. In some embodiments, the multicomponent complex comprises agglomerations of components, including proteins, such as monomer, dimer and trimer species, or other higher order agglomerations. Thus, the techniques described herein may be used to separate and analyse protein-protein interactions.

### Example: Neutralisation for Immunity Profiling

In this example, the aim is to extrapolate a property of an individual's immune profile based on measurements that are not directly querying that specific property. In this example, the specific application seeks to predict an individual neutralisation capacity for a mutant S1 protein, which has not been directly measured in a neutralisation assay, from the individual's neutralisation capacity of the wild-type S1 and the knowledge of how the mutation of S1 changes its affinity for the ACE2 receptor in vitro.

The assay is summarised in Equation 1 below:

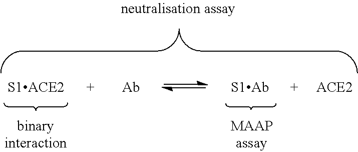

In this context, the following definitions are used:

Binary interaction is the “simple” F1 affinity measurement between two protein species, in this example two protein species, one of which is fluorescently labelled and the other one is titrated against it. The resulting size change as the labelled species forms a complex with the unlabelled one is detected through a change in the apparent hydrodynamic radius, measured via microfluidic diffusional sizing.

The acronym MAAP stands for Microfluidic Antibody Affinity Profiling. The MAAP assay is exemplified by an antigen (e.g. SARS-CoV-2 spike protein) being fluorescently labelled and a series of human serum dilutions are measured against it. By also varying the labelled species concentration, and not only the serum dilutions, both the affinity of antibodies in serum for the antigen and their concentration can be determined at the same time.

A neutralisation assay is a competition assay with three species playing a role in the reaction. Labelled ACE2 receptor is mixed with the SARS-CoV-2 spike protein and human serum dilutions are measured against it. As antibodies found in serum compete for binding the (unlabelled) spike protein, more and more of the labelled ACE2 receptor will be found unbound to the spike protein. This reduction in apparent hydrodynamic radius of the labelled species is detected, allowing for the quantification of the neutralising capacity of the antibodies found in serum. By varying the concentration/dilution of all three components of this reaction, both interaction affinities and also the antibody concentration in serum can be determined at the same time. However, the binary component interactions can also be measured independently, and the neutralisation can be performed at a fixed concentration of ACE2 and spike protein, with only the serum dilutions being varied.

The following experiments are included:
1. WT S1 binding to ACE2 measured in buffer—binary interaction
2. S1-N501Y binding to ACE2 measured in buffer— binary interaction

These assays are independent of individuals and only need to be performed once. They inform the general model for this interaction, independent of a specific individual. The data produced in these assays is fit to a binary model in order to determine the affinity, K_{D}, values for these interactions.
3. MAAP assay of WT S1 against patient serum
4. MAAP assay of S1-N501Y against patient serum
5. Neutralisation assay with WT S1, ACE2 and patient serum
6. Neutralisation assay with S1-N501Y, ACE2 and patient serum

Experiments 3 and 5 are the only experiments performed with the WT S1 and without any other mutants. The reason for performing both experiment 3 and experiment 5 is that there is a limited concentration range used in the neutralisation assay of experiments 5. In principle, by measuring many data points in the neutralisation assay across combination of different S1, ACE2 concentrations and serum dilutions it would be possible to achieve the same. Currently, the neutralisation assay fixes both the S1 and ACE2 concentrations and only varies the serum dilution. Going forward it should be possible to run assay 5 without first running assay 3. Similarly, going forward it should be possible to run assay 6 without running assay 4.

Using the data generated by above mentioned experiments 1, 3 and 5, a WT neutralisation model is used to determine the two K_{D} values and the antibody concentration in one fitting.

Following this, the K_{D} value from experiment 2 is exchanged into the neutralisation model in order that the changes to the binding to ACE2 of the mutation are taken into account. From this a neutralisation curve is predicted for the patient against the S1-N501Y without having to perform any additional measurements.

Experiments 4 and 6 are performed to validate the output of this modelling to check that the modelled data matches reality.

The key assumption involved in this procedure is that the mutation doesn't change the affinity of neutralising antibodies for the S1 protein. Typically, each mutation behaves similarly across different patients, in that it either has no effect on antibody binding or it has a similar affinity-reducing effect across the majority of patients. The model can, in principle account for either of the above mentioned scenarios. This hypothesis needs to be validated for each mutant on a set of in-house patient sera to have a reasonable degree of certainty that this key assumption holds.

Data from this example are shown in FIGS. 4 and 5A and 5B.

FIG. 4 shows a comparison of the “real” neutralisation model based on experiments 2, 4 and 6. This is shown in solid line with corresponding uncertainty or confidence illustrated in /// hatch. The dashed curve is a predicted model, based on a baseline WT model using experiments 1, 3 and 5, with the mutant S1/ACE2 affinity swapped in from experiment 2. The uncertainty or confidence in the predicted model is illustrated in \\\\ hatch.

Control experimental data is shown in FIGS. 5A and 5B. The same key is used as in FIG. 4 above. In FIG. 5A, the “real” neutralisation model based on experiments 1, 3 and is compared to a predicted model based only on 1 and 3. In FIG. 5B, the “real” neutralisation model based on 2, 4 and 6 is compared to a predicted model based only on 2 and 4. It is noted that the N501Y neutralisation data is quite noisy.

Referring to FIG. 6, there is provided a device 600 comprising a sample port 611 for loading a sample into the device 600. The device 600 further comprises, extending from the sample port 611, a sample channel 612 for flowing the sample comprising one or more bio-molecular components at a first flow rate into an elongate distribution channel 616. An auxiliary inlet port 613 is provided for loading an auxiliary fluid flow into an auxiliary channel 614. The auxiliary channel 614 is configured to introduce the auxiliary fluid flow at a second flow rate into the elongate distribution channel 616. The distribution channel 616 is configured to enable a lateral distribution of the components from the sample fluid flow into the auxiliary fluid flow after a steady state distribution is reached.

Two capillary channels 618 are provided downstream and in fluid communication with the distribution channel 616 such that at least a part of the steady state fluid flow that has been reached moves into each of the capillary channels 618. A portion of each of the capillary channels 618 is arranged in a serpentine or tortuous configuration 620. In other embodiments, not illustrated in the accompanying drawings, there may be more than two capillary channels provided downstream of the distribution channel.

An outlet port 626 is provided downstream and in fluid communication with the capillary channel 618. Detection and analysis of the component using one or more detectors can be carried out at the serpentine or tortuous region 620 of the capillary channels and/or within the outlet port 626.

In some instances, the detector (not shown in the accompanying drawings) can be configured to detect and measure at least one biophysical property of the or each bio-molecular component sequentially or simultaneously in each of the capillary channels 618 on a microfluidic chip. Additionally or alternatively, the detector may be configured to detect and measure at least one biophysical property of the or each component sequentially or simultaneously in each of the outlet ports 626 on the microfluidic chip.

A chip can be provided to include a plurality of microfluidic circuits 600. The plurality of microfluidic circuits or devices 600 may be arranged in parallel to each other. The microfluidic circuit or microfluidic device 600 can be capillary filled with one or more fluid flows such as a sample fluid flow and/or an auxiliary fluid flow. Multiple measurements can be done on the chip.

Referring to FIG. 7, there is provided an example of an instrument 700 including a user interface 702 configured to communicate the measurements of one or more biophysical property of one or more components in a fluid in each of a plurality of microfluidic chips 600 present on a chip plate 704.

The chip plate 704 including the plurality of microfluidic chips 600 is configured to be inserted into the instrument 700 which operates to detect which microfluidic chips 600 have been used on the chip plate 704. The instrument includes a display panel 706 configured to display this information to a user. The display provides information about each microfluidic chip 600. The information can be a binary indication as to whether or not each microfluidic chip 600 has been used and therefore whether or not it is available for the next experiment. In some instances, the display can also show an image of the plate 704 in use during experimentation.

The instrument 700 may also contain a reader module (not shown in the accompanying drawings) configured to detect and read a unique authentication indicium, such as a barcode, positioned on the chip plate 704. Other forms of unique authentication code can be used on the chip plate: for example, a unique sets of numbers, batch codes, QR codes or a combination of letters and numbers that are unique to each chip plate 704. Alternatively the authentication indicium may be stored on an NFC or RFID tag. If the chip plate 704 has passed its expiry date, the instrument can display this information to the user.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

It will further be appreciated by those skilled in the art that although the invention has been described by way of example with reference to several embodiments. It is not limited to the disclosed embodiments and that alternative embodiments could be constructed without departing from the scope of the invention as defined in the appended claims.

1. A system for developing a predictive immunity profile on the basis of the quantitative analysis of one or more samples from an individual, the system comprising:
a device configured to perform quantitative analysis of the interaction between one or more target species and one or more probes in solution on a fluid sample to provided quantitative analysis data;
a data store storing:
personal data relating to at least one individual;
at least one model of target/probe interaction;
processing circuitry configured to
access the data store and identify and retrieve data relevant to the sample;
receive quantitative analysis data of the sample from the device;
perform analysis to fit the model to the received quantitative analysis data;
extrapolate, through the model, to create a predictive immunity profile for the individual, and
update the data store with the quantitative analysis data.

2. The system according to claim 1, wherein the predictive immunity profile includes a date on which the patient's immunity is predicted to fall below a predetermined threshold.

3. The system according to claim 1, wherein the predictive immunity profile includes a confidence level that the individual will exhibit immunity to a different disease strain from the strain on which the vaccine was based.

4. The system according to claim 1, wherein the predictive immunity profile includes a confidence level that the individual will exhibit immunity to a different disease strain from the strain that the individual contracted.

5. The system according to claim 1, wherein the data store holds data on multiple individuals.

6. The system according to claim 5, wherein the data held in the data store on each individual includes one or more of the following: medical records, age, gender, weight, ethnicity, genetic information, vaccination history, disease state, disease severity, identity of medication prescribed and corresponding dosage regimen.

7. The system according to claim 1, wherein the data store further comprises stratification labels and wherein the processing circuitry is further configured to use the stratification labels when performing analysis to fit the received quantitative analysis data to the model.

8. The system according to claim 1, wherein the data store further comprises a general model and wherein the processing circuitry is further configured to use the general model when performing analysis to fit the received quantitative analysis to the model.

9. The system according to claim 1, wherein circuitry configured to perform said analysis comprises a machine learning algorithm.

10. The system according to claim 1, wherein the processing circuitry is further configured to update the personal data relating to the individual's sample analysed.

11. The system according to claim 1, wherein the quantitative analysis of the sample includes a measurement of affinity of a target/probe interaction.

12. The system according to claim 1, wherein the quantitative analysis of the sample includes a measurement of the concentration of a target species within the sample.

13. The system according to claim 1, wherein the quantitative analysis of the sample includes analysis of the heterogeneity of the sample.

14. The system according to claim 1, wherein the device comprises a microfluidic network configured to enable combination and distribution of a sample fluid and an auxiliary fluid to create a distributed sample and subsequent division of the distributed sample into two or more parts and measurement of at least one of the parts.

15. The system according to claim 1, wherein the distribution is created by one or more of diffusion, electrophoresis or magnetophoresis, thermophoresis, chromatography and isoelectric focusing.

16. The system according to claim 1, wherein the device is configured to divide the distributed sample into more than two parts and measurement is carried out on each divided part.

17. The system according to claim 1, wherein each predicted result generated by the system has an associated accuracy score.

18. The system according to claim 17, wherein the step of updating the data store includes updating the accuracy score.

19. The system according to claim 1, wherein the quantitative analysis of the device includes a fluorescence measurement.
